(19) United States
(12) Patent Application Publication
(10) Pub. No.: US 2017/0105876 A1
(43) Pub. Date: Apr. 20, 2017

(54) HEADACHE RELIEF EARPLUG

(71) Applicant: CIRRUS HEALTHCARE PRODUCTS, LLC, Cold Spring Harbor, NY (US)

(72) Inventors: Drew E. O'Connell, SR., Cold Spring Harbor, NY (US); Nicholas A. Petry, Manchester, VT (US); Lansing M. Lewis, III, Tallahassee, FL (US)

(21) Appl. No.: 15/050,114

(22) Filed: Feb. 22, 2016

Related U.S. Application Data
(60) Provisional application No. 62/243,756, filed on Oct. 20, 2015.

(51) Int. Cl. A61F 11/08

(52) U.S. Cl. CPC A61F 11/08; A61F 2011/085; A61F 2250/0069

(57) ABSTRACT

The present invention is directed to an earplug including a body having a first end, a second end and a longitudinal axis extending from the first end to the second end, a bore defined within the body and extending from the first end to the second end along the longitudinal axis of the body, and a pressure regulator positioned within the bore and providing an air flow rate of 3.4×10⁻⁶ to 7.8×10⁻⁵ cc/sec through the bore.

### CROSS-REFERENCE TO RELATED APPLICATIONS

This application claims priority to U.S. Provisional Appl. No. 62/243,756 filed Oct. 20, 2015, which is hereby incorporated by reference in its entirety.

### BACKGROUND OF THE INVENTION

1. Field of the Invention

The present invention relates to a barometric pressure regulating device used for regulating the rate of change of pressure inside an ear to minimize the pressure differential between the middle ear and a volume of the external ear cavity adjacent the ear drum. More particularly, the present invention is directed to ear plug adapted to fit tightly within the external ear cavity with no or minimal air leakage around the ear plug, and disposed within the ear plug is a pressure regulator with a slow leak rate, that may preferably be made of a porous material.

2. Description of Related Art

The middle ear is an air-filled cavity that connects to the outside environment via the Eustachian tube. Under normal conditions, there is no difference between the air pressure in the outside environment and the middle ear. This condition is illustrated in FIG. 1 which is a schematic drawing of the ear, and shows that under normal conditions there is no pressure difference across the eardrum. However, when there is a change in air pressure, such as when there is a change in barometric pressure due to weather conditions, change in elevation, etc., there is a difference in the air pressure between the middle ear and the outside environment. Under conditions in which such a pressure differential exists, the Eustachian tube acts as a valve between the outside environment and the middle ear. In this capacity, the Eustachian tube opens for a fraction of a second in response to swallowing or yawning, allowing airflow through it so that the pressure differential between the outside environment and the middle ear equilibrate.

During a decrease in barometric pressure the air pressure in the outside environment, that is, the environment outside the middle ear, is less than that of the pressure inside the middle ear. Under these conditions, the positive relative pressure in the middle ear forces air out of the Eustachian tube thereby lowering the pressure inside the middle ear to the same pressure as the outside environment. If the Eustachian tube is blocked, as described more fully below, the positive pressure in the middle ear applies pressure to the ear drum, as shown schematically in FIG. 2, causing it to bow outward toward the external ear cavity.

Conversely, during an increase in barometric pressure, the air pressure in the outside environment is greater than that of the pressure inside the middle ear. Under these conditions, the negative relative pressure in the middle ear causes airflow from the outside environment through the Eustachian tube and into the middle ear, thereby increasing the pressure inside the middle ear to the same pressure as the outside environment. Again, under condition in which the Eustachian tube is blocked, the negative pressure in the middle ear causes deformation of the ear drum, bowing it inward toward the middle ear, as shown schematically in FIG. 3.

Under normal circumstances, when there is no or little blockage of the Eustachian tube, as the pressure differential increases across the ear drum between the middle ear and outside environment, voluntary swallowing and/or chewing releases the pressure through the Eustachian tube.

A barometric pressure headache is a type of migraine headache that is caused by a change in atmospheric air pressure and is characterized by a pounding headache centered on the front of the head and sinus area. Approximately 72% of migraines are related to barometric pressure changes. Barometric pressure is the weight of air pressing against the Earth, and is called barometric pressure because such pressure is measured by barometers. Barometric pressure can be affected by weather, in that an approaching storm causes barometric pressure to drop, which is usually when a barometric pressure headache will form. Some treatments include taking pain medication and taking steps to counteract the pressure change by lowering blood pressure. Barometric changes generally range from 31 inches of mercury to 29 inches of mercury with a base pressure of 29.92. The psi differential for sea level barometric pressure reading at the high and low readings is 0.982 psi. While moving from sea level to a much higher altitude, e.g. hiking, skiing, flying when barometric pressure is high will greatly increase the psi change as a result of gaining altitude. However, when not changing altitude, the pressure differential between potential high and low barometric pressures is small. Even though this pressure differential may be relatively small, it may still be significant enough to induce barometric pressure headaches. Therefore, what is needed is a means for reducing the severity of pain and duration of pain associated with barometric pressure headaches.

While prior devices, such as the earplugs discussed in U.S. Pat. No. 5,467,784, which is hereby incorporated by reference in its entirety, provide for pressure regulation of the ear canal, such prior devices are intended for rapid changes in pressure associated with commercial air travel. These prior devices do not provide sufficient adaptation when the barometric pressure changes are relatively small or slow to occur. Furthermore, these prior devices are used for short periods of time due to the rapid changes in pressure associated with such activities as commercial air travel, but are generally not suitable for long durations of use.

Therefore, what is needed in order to provide for the reduction of the severity of pain and duration of pain associated with a barometric pressure headache is an earplug that provides for reducing the speed at which the earplug allows the pressure on the ear drum to be equitized, and can be comfortably worn for long period of time.

### SUMMARY OF THE INVENTION

The present invention is designed to overcome the above noted limitations that are attendant upon the use of conventional earplugs and, toward this end, it contemplates the provision of a novel earplug that provides for regulated flow of air through the earplug to allow for mitigation of the effects caused by gradual barometric pressure changes when the earplug is installed in a person's ear canal and is sufficiently comfortable so as to be worn for long enough durations during the gradual barometric pressure changes.

It is an object of the present invention to provide a method of using an earplug in the treatment and/or prevention of barometric pressure headaches.

It is further an object of the present invention to provide an earplug and a method of using such earplug to counteract the effects of barometric pressure changes, such as the effects of barometric pressure headaches caused from barometric pressure changes.

It is yet another object of the present invention to provide an earplug and a method of using such earplug that reduces the severity of pain and duration of pain caused by a barometric pressure headache.

It is still another object of the present invention to provide an earplug and a method of using such earplug that also is configured to allow for sound attenuation to further help with mitigating headache discomfort.

It is yet another object of the present invention to provide an earplug that is configured to be comprised of a soft material, such as silicone, in order to be comfortable enough to be worn for long periods of time, e.g. 3 to 24 hours, but resilient enough to still perform the functions associated with a headache relief earplug.

It is an object of this invention to regulate the rate of airflow through an ear plug when it forms an air tight pressure seal in the ear canal.

It is a further object of this invention to reduce the rate of pressure change in an external ear canal to reduce the pain and discomfort caused by pressure differences across the ear drum, such as the pain and discomfort caused by a barometric pressure headache.

Accordingly, the headache relief earplug of the present invention is designed to make an air tight seal in the external ear canal and to regulate the rate of change in air pressure between the external ear canal and the middle ear when a user is exposed to changes in barometric atmospheric pressure. By so doing, the plug can delay the build-up of an air pressure differential between the external ear canal and the middle ear, which are separated by the ear drum. As a result, the user, even with a partially blocked Eustachian tube, has an extended period of time to equalize the barometric pressure differential between the middle ear and the external environment. Normally, these differentials are equalized by the Eustachian tube, which vents the middle ear into the throat. The pressure regulating ear plug creates an enclosed environment between the outside environment and the ear drum. Over time, the headache relief earplug slowly leaks air into or out of the volume immediately exterior to the ear drum, so that the pressure in said volume equilibrates with the pressure outside of the earplug. This delay increases the time available for the Eustachian tube to perform its function of equilibrating the middle ear pressure with the pressure in the external environment.

The headache relief earplug controls the rate at which the barometric pressure changes in the external ear canal adjacent the ear drum. The Eustachian tube then has more time to respond to the pressure changes thereby reducing discomfort. The amount of additional time the Eustachian tube has depends on the porosity of the regulating element inside the ear plug the ear plug of the present invention is an elongated tube of the type typically used for sound attenuation, with a slow leak porous medium disposed within the tube. Examples of such porous media include porous metal material and porous ceramic material, and the ceramic material is the presently preferred material for pressure regulation. Thus, the system can increase substantially the time required to equalize the pressure in the ear canal to the pressure in the environment in which the user is subjected. Air flow preferred for this application of barometric pressure change control is projected to be 3.4×10⁻⁶-7.8×10⁻⁵ cc/sec. The filter porosity for the pressure regulating device used with the headache relief earplug according to an exemplary embodiment of the present invention may preferably be 1.3-2 microns.

The present invention is also effective for the purpose of sound attenuation by default being a blockage in the ear canal and absorbing sound waves as they enter the ear canal. This is an added benefit to the plugs use in mitigating the intensity of a barometric pressure headache.

In accordance with another exemplary aspect of the present invention, the material used for forming the headache relief earplug may be comprised of a soft yet resilient material, for example a soft molded silicone. Preferably, the soft molded silicone may have a durometer between 60 and 64 on the OO scale.

Both sound attenuation and comfort of the headache relief earplug due to its construction with a soft material may also help to mitigate headache discomfort if the headache has already begun.

### BRIEF DESCRIPTION OF THE SEVERAL VIEWS OF THE DRAWINGS

For a fuller understanding of the nature and object of the present invention, reference should be had to the following detailed description taken in connection with the accompanying drawings, in which:

FIG. 1 is a schematic view of an ear in which there is no pressure differential between the external environment and the middle ear;

FIG. 2 is a schematic view of an ear in which there is a pressure differential between the external environment and the middle ear wherein the pressure in the middle ear is greater than the pressure in the external environment, and wherein the Eustachian tube is blocked;

FIG. 3 is a schematic view of an ear in which there is a pressure differential between the external environment and the middle ear wherein the pressure in the middle ear is less than the pressure in the external environment, and wherein the Eustachian tube is blocked;

FIG. 4 is a schematic view of an ear in which the headache relief earplug of the present invention is disposed, and in which there is a pressure differential between the external environment and the middle ear, the pressure being less in the middle ear than in the exterior environment, illustrating a change in barometric pressure, and wherein the Eustachian tube is blocked;

FIG. 5 is a schematic view of an ear in which the headache relief earplug of the present invention is disposed, and in which there is a pressure differential between the external environment and the middle ear, the pressure being greater in the middle ear than in the exterior environment, illustrating a change in barometric pressure, and wherein the Eustachian tube is blocked;

FIG. 6 is a perspective view of an exemplary embodiment of the headache relief earplug according to the present invention; and

FIG. 7 is a cross-sectional view of the exemplary embodiment of the headache relief earplug according to the present invention taken through line 7-7 of FIG. 6.

### DETAILED DESCRIPTION OF THE INVENTION

The present invention now will be described more fully hereinafter with reference to the accompanying figures, in which exemplary embodiments of the invention are shown. The invention may, however, be embodied in many different forms and should not be construed as limited to the embodiments set forth herein.

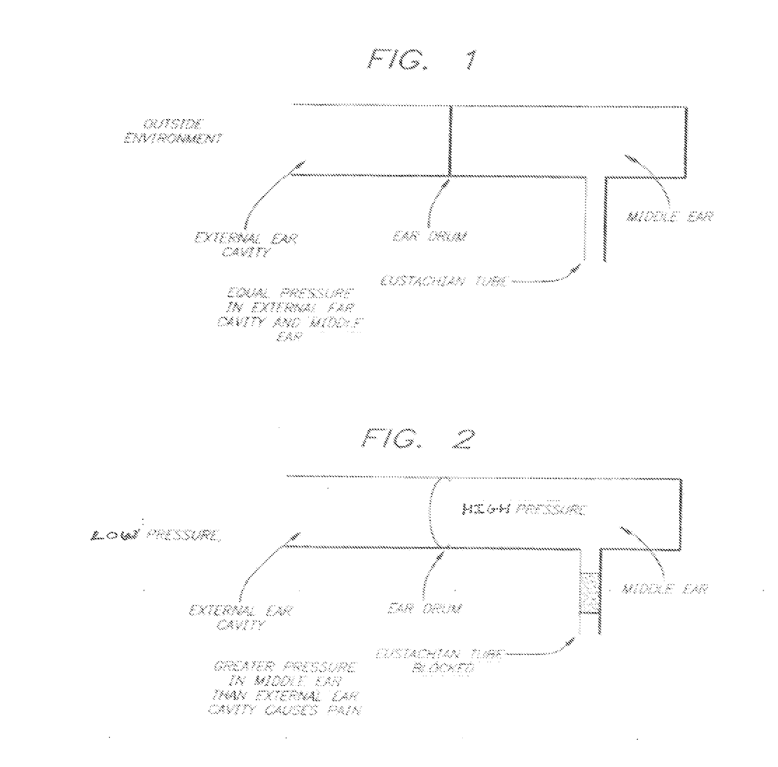

An exemplary embodiment of the present invention is illustrated schematically in FIGS. 4 and 5. FIG. 5 is a schematic illustration of the operation of the headache relief earplug of the present invention under conditions comparable to those of FIG. 2, except for the use of the exemplary embodiment of the headache relief earplug according to the present invention. More specifically, FIG. 5 depicts the effect of a decrease in barometric pressure. In FIG. 5, the outside environment has a lower barometric pressure, for example 29 inches of mercury or lower (14.243 Psia or less), than the pressure inside the middle ear 28, which is still at the barometric pressure, for example 29.92 to 31 inches of mercury (14.695 to 15.225 Psia), before the decrease of barometric pressure. The pressure inside the middle ear is a result of the middle ear being pressurized and filled with air prior to the decrease in barometric pressure, and not being able to equilibrate to the current ambient pressure because the Eustachian tube 26 is blocked, schematically depicted at 27, so that the air in the middle ear 28 cannot escape through the Eustachian tube, or at least not at the desired rate sufficient to cause the desired depressurization of the middle ear.

However, because of the installation of the headache relief earplug 24, which is schematically depicted in FIG. 5, the pressure in the volume between the ear drum 20 and the headache relief earplug 24 remains at the prior barometric pressure before the decrease in barometric pressure, and this prior barometric pressure is the same as the pressure in the middle ear. The headache relief earplug 24 is adapted to slowly release air in the volume 30 to the outside environment through the external ear canal 22, and as shown, airflow in direction A illustrated by the arrows occurs. Similarly, provided that there is only partial blockage of the Eustachian tubes, airflow through the blockage 27 travels in the direction depicted by arrow B, so that the pressure in the middle ear equilibrates with the current ambient barometric pressure as it exists in the outside environment.

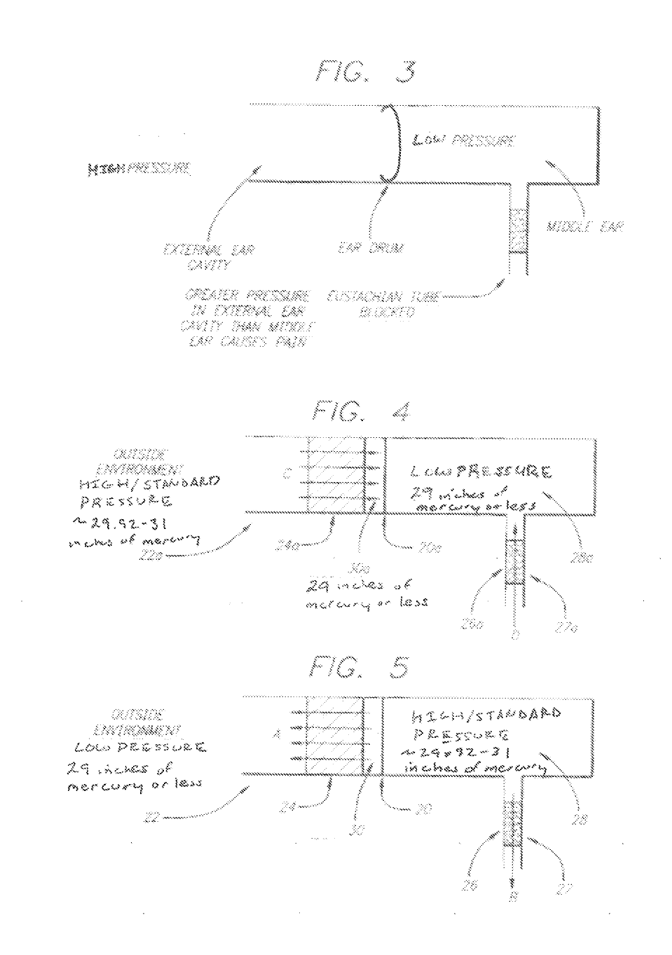

Similarly, FIG. 4 is a schematic illustration of the operation of the headache relief earplug of the present invention under conditions comparable to those of FIG. 3, except for the use of the exemplary embodiment of the headache relief earplug according to the present invention. More specifically, FIG. 4 depicts the effect of an increase in barometric pressure. In FIG. 4, the outside environment has a higher barometric pressure, for example 29.92 to 31 inches of mercury (14.695 to 15.225 Psia), than the pressure inside the middle ear 28a, which is still at the barometric pressure, for example 29 inches of mercury or lower (14.243 Psia or less), before the increase in barometric pressure. The pressure inside the middle ear is a result of the middle ear being pressurized and filled with air prior to the increase in barometric pressure, and not being able to equilibrate to the current ambient barometric pressure of the outside environment because the Eustachian tube 26a is blocked, schematically depicted by blockage 27a, so that the middle ear 28a cannot draw in air through the Eustachian tube, or at least not at the desired rate sufficient to cause the desired pressurization of the middle ear. However, because of the installation of the headache relief earplug 24a, which is schematically depicted in FIG. 4, the pressure in the volume 30a between the ear drum 20a and the pressure regulating earplug 24a remains at the prior barometric pressure before the increase in barometric pressure, and this prior barometric pressure is the same as the pressure in the middle ear. The headache relief earplug 24a is adapted to slowly permit the inflow of air into the volume 30a through the external ear canal 22a and as shown, airflow in direction C illustrated by the arrows occurs. Similarly, provided that there is only partial blockage of the Eustachian tubes, airflow through the blockage 27a travels in the direction depicted by arrow D, so that the pressure in the middle ear equilibrates with the current ambient pressure as it exists in the outside environment.

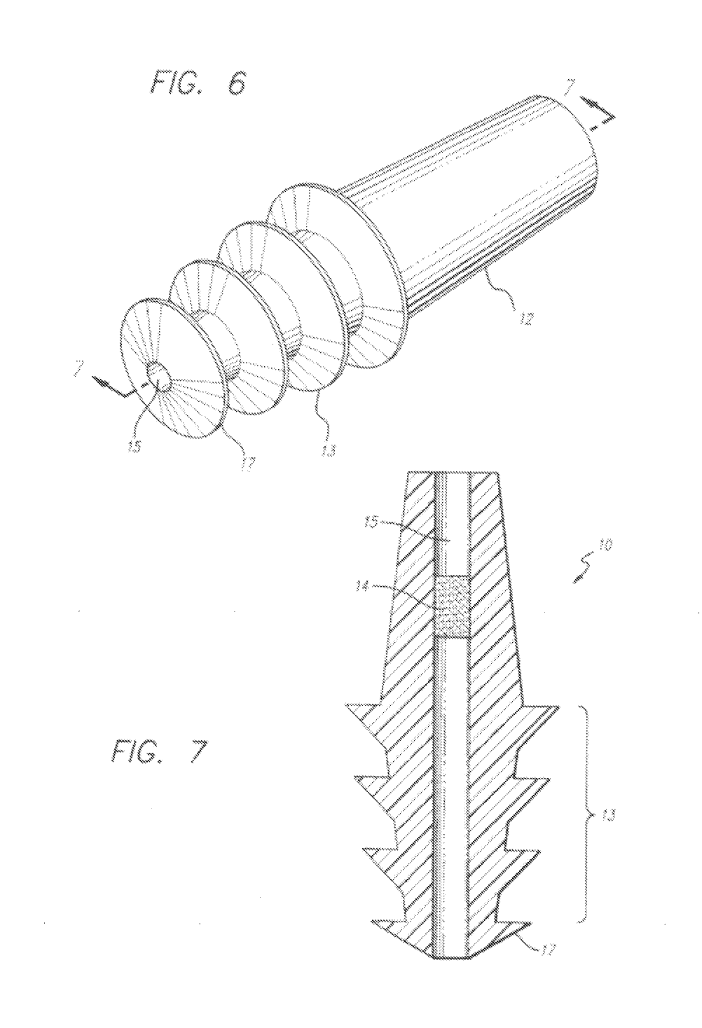

An exemplary embodiment of the invention is illustrated in FIGS. 6 and 7. FIG. 7 shows an enlarged cross-sectional view of the exemplary embodiment of the headache relief earplug 10 of the present invention with each of the components identified. The headache relief earplug 10 has a body 12 shaped generally like a conventional sound attenuating ear plug body comprising a ribbed neck section 13 of the headache relief earplug 10. The ribbed neck section 13 provides an air tight seal with the walls of the ear canal when the headache relief earplug 10 is in use. The seal is important to ensure that the pressure regulation is controlled by the pressure regulator 14 and is not affected by a poorly sealed ear plug. There is a bore 15 extending through the headache relief earplug 10 to permit airflow regulated by the pressure regulator 14 therethrough. The pressure regulator 14, which is preferably made of porous metal or porous ceramic, and most preferably, porous ceramic material, permits slow air leakage therethrough, preferably in the range of 3.4×10⁻⁶ to 7.8×10⁻⁵ cc/sec. FIG. 6 shows a perspective view of the subject invention in its preferred embodiment with the plurality of ribs 17 providing a secure and leakage resistant means of retaining the earplug in the ear and preventing any air leakage except through the pressure regulator 14.

The pressure regulator 14 may be made from a porous ceramic material, and the porous ceramic material may preferably be comprised of 73.9% by weight of Al₂O₃, 24.6% by weight of SiO₂, 0.1% by weight of CaO, 0.1% by weight of MgO, 0.4% by weight of Fe₂O₃, 0.4% by weight of TiO₂, 0.3% by weight of K₂O and 0.2% by weight of Na₂O. The porous ceramic material may also preferably be P-3-C CoorsTek material available from CoorsTek, Inc. of Golden, Colo. The pressure regulator 14 may be made by combining the porous ceramic material with a bonding agent and forming the combination into a small right circular cylinder approximately 0.125 inches (3.18 mm) long with a diameter of 0.083 inches (2.1 mm). Once the cylinder is formed it is heated until the material fuses together and forms the solid pressure regulator 14. The porosity of the ceramic is controlled by adjusting the particle size, bonding agent, and controlling the curing temperature or the heating profile. Preferable, the pressure regulator 14 will have a porosity of between approximately 1.3 and 2 microns. The pressure regulator 14 may then be forced into the bore 15 of the headache relief earplug 10 which has an inside diameter of 0.078 inches (1.98 mm). The interference fit provides an air-tight seal between the pressure regulator 14 ceramic and the bore 15 of the headache relief earplug 10.

The body 12 of the headache relief earplug 10 may be made from any suitable material used for the manufacture and/or construction of earplugs. Preferably, the material used to construct the body 12 should be sufficiently air-tight so that air only passes through the pressure regulator 14, and should also be sufficiently resilient so as to be capable of forming at least a substantially air-tight seal with a user's ear canal. Even more preferably, the material may be a soft molded silicone having a durometer of between about 60 to 64 on the OO scale. It is understood that the OO scale has a spherical radius of 1.20 mm, a diameter of 2.40 mm, an extension of 2.54 mm and a spring force of 113 gf (1.11N).

An exemplary method of using the exemplary headache relief earplug 10 for the treatment of headaches, including barometric pressure headaches, will be discussed with reference to FIGS. 6 and 7. The headache relief earplug 10 may be inserted into the ear canal and/or canals of a user, preferably both ear canals of a user, either upon the onset or prior to the onset of the symptoms of a headache. The user of the headache relief earplugs 10 may be aware of impending conditions that may result in the onset of headache symptoms, such as a change in barometric pressure, and the headache relief earplug 10 may be inserted into the user's ear canals to reduce the likelihood of experiencing such symptoms. Alternatively, the headache relief earplugs 10 may be inserted into the user's ear canal(s) after headache symptoms have begun, and the headache relief earplugs 10 are configured to reduce the duration and severity of the headache symptoms. The headache relief earplugs 10 may be worn in the user's ear canal(s) for any duration of time, and preferably the amount of time required for equilibration of pressure external to the ear canal(s) with pressure internal to the ear canal(s), such as the pressure that exists within one or more sinuses of the user. This amount of time may vary from 3 to 24 hours, but it is understood that the present invention is not limited to any particular amount of time regarding the duration the earplugs are left within the user's ear canal(s). In the event that the user has inserted the headache relief earplugs 10 within the ear canal(s) prior to the onset of headache symptoms, and no headache symptoms are experience, the user may then leave the headache relief earplugs 10 within the ear canal(s) until the conditions which were likely to have caused headache symptoms have passed. The headache relief earplugs 10 should preferably be inserted into the user's ear canal(s) a sufficient depth so as to allow at least one the plurality of ribs 17 to form an airtight seal with the ear canal(s). In this manner, the exchange of air occurs through the pressure regulator 14 so that the leak rate of the pressure regulator 14 adjusts for changes in pressure that may result in the onset or worsening of headache symptoms.

It will thus be seen that the objects set forth above, among those made apparent from the preceding description, are efficiently attained and, since certain changes may be made in the above article without departing from the scope of this invention, it is intended that all matter contained in this disclosure or shown in the accompanying drawings, shall be interpreted, as illustrative and not in a limiting sense. It is to be understood that all of the present figures, and the accompanying narrative discussions of corresponding embodiments, do not purport to be completely rigorous treatments of the invention under consideration. It is to be understood that the above-described arrangements are only illustrative of the application of the principles of the present invention. Numerous modifications and alternative arrangements may be devised by those skilled in the art without departing from the scope of the present invention.

What is claimed is:

1. An earplug, comprising:
a body having a first end, a second end and a longitudinal axis extending from the first end to the second end;
a bore defined within the body and extending from the first end to the second end along the longitudinal axis of the body; and
a pressure regulator positioned within the bore and providing an air flow rate of 3.4×10⁻⁶ to 7.8×10⁻⁵ cc/sec through the bore.

2. The earplug according to claim 1, wherein the earplug is configured to form an airtight seal with an ear canal when the earplug is inserted into the ear canal.

3. The earplug according to claim 1, wherein the body is consists primarily of an airtight material.

4. The earplug according to claim 1, wherein the pressure regulator is comprised of a porous material.

5. The earplug according to claim 4, wherein the porous material has a porosity of 1.3 to 2 microns.

6. The earplug according to claim 4, wherein the porous material is a porous ceramic material.

7. The earplug according to claim 2, wherein the body comprises a ribbed neck section comprising at least one rib positioned substantially concentrically around the longitudinal axis of the body, and configured to retain the earplug in the ear canal and prevent any air leakage between the earplug and the ear canal.

8. The earplug according to claim 1, wherein the body is comprised of a material having a durometer of between about 60 to 64 on the OO scale.

9. The earplug according to claim 1, wherein the body is comprised of silicone.

10. The earplug according to claim 1, wherein the pressure regulator is positioned within the bore so as to have an airtight seal between the pressure regulator and the body of the earplug.

11. A method of treating a headache, comprising:
inserting an earplug into an ear canal, and
leaving the earplug in the ear canal for a period of between about 3 to 24 hours,
wherein the earplug comprises:
a body having a first end, a second end and a longitudinal axis extending from the first end to the second end;
a bore defined within the body and extending from the first end to the second end along the longitudinal axis of the body; and
a pressure regulator positioned within the bore and providing an air flow rate of 3.4×10⁻⁶ to 7.8×10⁻⁵ cc/sec through the bore.

12. The method according to claim 11, wherein treating the barometric pressure headache comprises reducing the duration or severity of pain associated with the barometric pressure headache.

13. The method according to claim 11, wherein the earplug is configured to form an airtight seal with an ear canal when the earplug is inserted into the ear canal.

14. The method according to claim 11, wherein the headache is a barometric pressure headache.

15. The method according to claim 11, wherein the ear canal is connected to at least one sinus, and the earplug is configured to facilitate the equilibration of pressure within the at least one sinus and pressure external to the ear canal during the duration the earplug is left in the ear canal.

16. The method according to claim 11, wherein the body of the earplug is comprised of a material having a durometer of between about 60 to 64 on the OO scale.

17. The method according to claim 11, wherein the body of the earplug comprises a ribbed neck section comprising at least one rib positioned substantially concentrically around the longitudinal axis of the body, and configured to retain the earplug in the ear canal and prevent any air leakage between the earplug and the ear canal.

18. The method according to claim 11, wherein the pressure regulator of the earplug is comprised of a porous material.

19. The method according to claim 18, wherein the porous material has a porosity of 1.3 to 2 microns.

20. The method according to claim 18, wherein the porous material is a porous ceramic material.
